(19) United States
(12) Plant Patent Application Publication
(10) Pub. No.: US 2025/0374839 P1
(43) Pub. Date: Dec. 4, 2025

(54) Floribunda Rose Plant Named 'KORpoplol'

(50) ;

(71) Applicant: W. KORDES' SÖHNE Rosenschulen GmbH & Co KG, Klein Offenseth-Sparrieshoop (DE)

(72) Inventor: Wilhelm-Alexander Kordes, Klein Offenseth-Sparrieshoop (DE)

(73) Assignee: W. KORDES' SÖHNE Rosenschulen GmbH & Co KG, Klein Offenseth-Sparrieshoop (DE)

(21) Appl. No.: 19/217,189

(22) Filed: May 23, 2025

Related U.S. Application Data
(60) Provisional application No. 63/654,248, filed on May 31, 2024.

(51) Int. Cl. A01H 6/74; A01H 5/02

(52) U.S. Cl. CPC A01H 6/749; A01H 5/02
USPC PLT/142

(57) ABSTRACT

A new and distinct variety of floribunda rose plant, referred to by its cultivar name, ‘KORpoplol’, is described. The new variety forms in abundance on a substantially continuous basis orange-yellow-colored striped blossoms. The growth habit is bushy and compact. Semi-glossy, dark green foliage is formed. Additionally, the new variety is particularly well suited for growing as distinctive ornamentation in the landscape.

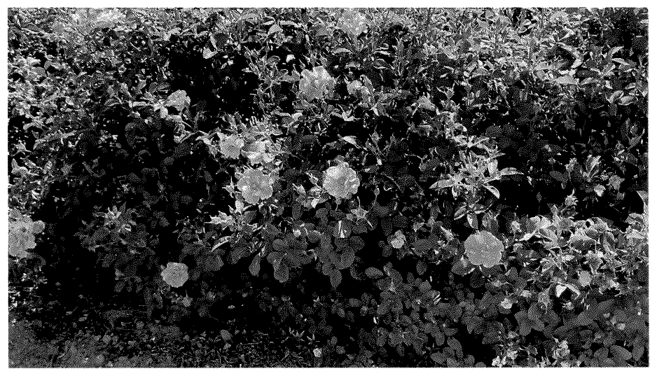

### CROSS REFERENCE TO RELATED APPLICATIONS

This application claims priority to U.S. Provisional Patent Application No. 63/654,248, which was filed on May 31, 2024, the contents of which are hereby incorporated by reference for all purposes.

Latin name of genus and species of plant claimed: Rosa hybrida.

Variety denomination: ‘KORpoplol’.

### STATEMENT REGARDING PRIOR DISCLOSURES BY THE INVENTOR

The first sale or offer for sale of the new variety was June 2023, on the KORDES website. The first sale or offer for sale of the new variety was by the inventor or another who obtained the new variety directly or indirectly from the inventor. No plants of the new variety have been sold in this country or anywhere in the world, nor has any disclosure of the new plant been made, more than one year prior to the effective filing date of this application, and such sale or disclosure within one year was either derived directly or indirectly from the inventor.

### BACKGROUND OF THE INVENTION

The new variety of floribunda rose plant of the present invention was created by controlled breeding in May 2012 in Sparrieshoop, Germany by artificial pollination wherein two parents were crossed which previously had been studied in the hope that they would contribute the desired characteristics. The female parent (i.e., the seed parent) was an unnamed breeder seedling (not patented). The male parent (i.e., the pollen parent) of the new variety was ‘KORrosobi’ (U.S. Plant Pat. No. 31,256).

The parentage of the new variety can be summarized as follows:

unnamed breeder seedling x ‘KORrosobi’

The seeds resulting from the above pollination were sown and small plants were obtained which were physically and biologically different from each other. Selective study resulted in the identification of a single plant of the new variety.

The new variety has been found to undergo asexual propagation at Wasco, California and Cochranville, Pennsylvania by a number of routes such as vegetative cuttings. Asexual propagation techniques in Wasco, California and Cochranville, Pennsylvania, such as vegetative cuttings, have shown that the characteristics of the new variety are homogeneous, stable, and strictly transmissible by such asexual propagation from one generation to another. Accordingly, the new variety undergoes asexual propagation in a true-to-type manner.

### SUMMARY OF THE INVENTION

It was found that the new variety of floribunda rose plant of the present invention possesses the following combination of characteristics:
(a) abundantly and substantially continuously forming orange-yellow-colored striped blossoms,
(b) exhibiting a bushy and compact growth habit, and
(d) forming semi-glossy, dark green foliage.

The new variety well meets the needs of the horticultural industry. It can be grown to advantage as distinctive ornamentation in parks, gardens, public areas, and residential landscapes. Accordingly, it is particularly well suited for growing in the landscape.

The new variety can be readily distinguished from its ancestors. More specifically, the new variety displays more glossy foliage and improved disease resistance compared to the unnamed breeder seedling parent (i.e., the seed parent) and the new variety exhibits orange-yellow-colored striped blossoms, whereas the seed parent exhibits yellow-pink-colored striped blossoms. Additionally, the new variety displays an orange-yellow-colored striped blossom, a lower petal count, and a more compact growth habit compared to the ‘KORrosobi’ variety (i.e., the pollen parent), which displays orange-colored blossoms. Moreover, the new variety can be readily distinguished from non-parental related similar varieties. For example, the new variety displays flowers with approximately 5 petaloids and exhibits semi-glossy foliage, whereas the ‘Meitalrea’ variety (U.S. Plant Pat. No. 26,082) displays flowers commonly without petaloids and exhibits glossy foliage.

The new variety has been named the ‘KORpoplol’ variety.

### BRIEF DESCRIPTION OF THE PHOTOGRAPHS

The accompanying photograph shows, as nearly true as it is reasonably possible to make the same in a color illustration of this character, a typical specimen of the new variety and blossoms of the new variety. The illustrated rose plant of the new variety was approximately two years of age and was grown outdoors in a field on understock in Wasco, California, U.S.A. in June 2022.

Drawing—illustrates a specimen of the plant displaying flowers at varying points of opening.

### DETAILED BOTANICAL DESCRIPTION

The chart used in the identification of colors is that of The Royal Horticultural Society (The R.H.S. Colour Chart, 2015 edition). The terminology which precedes reference to the chart has been added to indicate the corresponding color in more common terms and The R.H.S. Colour Chart designation used herein represents the closest color observed on the majority of the specified botanical feature. The description is based on a two-year-old specimen of the new variety, observed during May 2024, while growing on its own roots in a three-gallon container in a nursery in Cochranville, Pennsylvania.
Botanical classification: Rosa hybrida cultivar ‘KORpoplol’.
Commercial classification: Floribunda Rose.
Plant:
   Habit.—Bushy and compact.
   Height.—Approximately 90.0 cm on average from the top of the soil plane.
   Width.—Approximately 90.0 cm on average.
Branches:
   Stem color.—Old wood: Yellow-Green Group 146A. — young stems: Yellow-Green Group 144A.
   Length.—Main stems: typically 80.0 cm to 90.0 cm. — secondary stems: approximately 45.0 cm on average.
   Diameter.—Main stems: approximately 2.5 cm on average. — secondary stems: approximately 8.0 mm on average.
   Texture.—Smooth.
   Prickles.—Amount: excessive; 25 to 30 prickles per internode. — young prickles: length is approximately 3.0 mm on average; width is approximately 2.8 mm on average at point of attachment; and color is Yellow-Green Group N148C. — old prickles: length is approximately 6.0 mm on average; width is approximately 4.0 mm on average at point of attachment; and color is Greyed-Orange Group 165B.
Leaflets:
   Number.—3, 5, and 7.
   Shape.—Ovate; apex is acute to acuminate; and base is cuneate.
   Arrangement.—Odd-pinnate.
   Margin.—Serrate.
   Undulation.—Moderate.
   Glossiness of upper side.—Semi-glossy.
   Texture.—Upper surface: smooth. — under surface: smooth.
   Texture.—pinnate.
   Size.—Terminal leaflet: length is approximately 4.2 cm on average; width is approximately 3.0 cm on average. — lower leaflets: length is typically 2.0 cm to 3.5 cm; width is typically 2.0 cm to 2.5 cm. — 5-Leaflet leaf: length is approximately 8.5 cm on average; width is approximately 5.5 cm on average.
Foliage:
   Young foliage.—Upper surface color: Green Group 143A with indistinguishable venation. — under surface color: Green Group 143C and some Red-Purple Group 60A around the margins with indistinguishable venation.
   Old foliage.—Upper surface color: Green Group 139A with indistinguishable venation. — under surface color: Yellow-Green Group 147B with indistinguishable venation.
   Petiole.—Texture: upper surface is hispid; under surface is hispid and thorny. — length: approximately 2.7 cm on average. — width: approximately 1.0 mm on average. — upper surface color: Green Group 137B. — under surface color: Green Group 138A.
   Rachis.—Length: approximately 1.5 cm on average. — width: approximately 1.0 mm on average. — upper surface color: Green Group 139C. — under surface color: Green Group 143A.
   Stipules.—Length: approximately 1.0 cm on average. — width: approximately 2.0 mm on average. — margin: entire to erose. — upper surface color: Green Group 143A. — lower surface color: Green Group 137D.
Inflorescence:
   Number of flowers.—About 40 to 45 blooms on average on a plant at once.
   Number of blossoms per stem or in a cluster.—Typically between 1 and 6 blooms per stem on average.
   Peduncle.—Color: Green Group 143A. — diameter: approximately 2.0 mm on average. — length: approximately 5.0 cm on average. — surface texture: hispid.
   Sepals.—Number: 5. — upper surface color and texture: Green Group 143C, covered in short pubescence. — under surface color and texture: Green Group 143C, puberulent. — size: length is approximately 2.4 cm on average; width is approximately 8.0 mm on average. — margin: entire with extensions on most sepals measuring approximately 6.0 mm in length and 1.0 mm in width on average. — apex: acute to aristate. — base: truncate as it joins the receptacle.
   Buds.—Shape: ovoid. — size: length is approximately 2.0 cm on average; width is approximately 1.2 cm on average. — color (when opening): Yellow Group 13B swirled with Orange-Red Group N34B.
   Flower.—Form: double, cuplike. — profile: concave as it opens, and then outer petals are convex as the bloom ages and opens further. — diameter: approximately 6.0 cm on average. — height: approximately 2.5 cm on average. — duration: on the plant approximately 10 to 12 days. — petal color when first and fully opened: upper surface is striped, Yellow Group 13C and Orange-Red Group 31C with a basal spot of Yellow Group 13A; under surface is striped, Orange-Red Group 31D and Yellow Group 13C with a basal spot of Yellow Group 13A. — petal color at end of bloom: upper surface is striped, Orange-Red Group 32D and Yellow Group 4B with a basal spot of Yellow Group 13A; under surface is striped, Orange-Red Group 32D and Yellow Group 13C with no distinct basal spot. — petals reflex one by one: yes.
   Fragrance.—Very slight sweet scent.
   Petal.—Number: 10 to 12 on average. — drop: good. — length: typically 3.0 cm to 4.0 cm. — width: typically 3.0 cm to 4.0 cm. — overall shape: broadly obovate. — margin: entire with moderate undulation. — apex shape: rounded to slightly cuspidate. — base shape: cuneate. — texture: upper and under surfaces are smooth.
   Petaloids.—Number: approximately 5 on average. — length: approximately 2.0 cm on average. — width: approximately 8.0 mm on average. — color: largely Orange-Red Group 32D with slight striping of Yellow Group 13B with a basal spot of Yellow-Orange Group 14A. — texture: upper surface is smooth; under surface is smooth. — margin: entire. — shape: spatulate to slightly obovate at apex. — apex: obovate. — base: truncate.
   Stamen.—Number: approximately 107. — anthers: number is approximately 107; color is Yellow-Orange Group 23A; length is approximately 4.0 mm on average; and shape is oval. — filaments: length is approximately 6.0 mm on average and color is Yellow-Orange Group 21A.
   Pistils.—Arrangement: separate and free. — number: approximately 80. — color: Red Group 37A. — style: length is approximately 7.0 mm on average and color is Red Group 37A. — stigma: diameter is approximately 1.0 mm on average; and shape is fan shaped.
   Receptacle.—Diameter: approximately 6.0 mm on average. — depth: approximately 6.0 mm on average. — shape: urn shaped. — color: Yellow-Green Group 144B. — surface texture: smooth.
   Pollen.—Amount: sparse. — color: Yellow-Orange Group 20A.
   Hips/seed.—None observed.
Development:
   Vegetation.—Semi-glossy, dark green, and vigorous.
   Blossoming.—Abundant and substantially continuous from spring through frost; typically, in bloom outdoors from May to November in Southeastern Pennsylvania.
   Resistance to diseases.—Very good resistance to powdery mildew (Podosphaera pannosa) and rust (Phragmidium tuberculatum).
   Hardiness.—Hardy to USDA Zone 6.

The new ‘KORpoplol’ variety has not been observed under all possible environmental conditions to date. Accordingly, it is possible that the phenotypic expression may vary somewhat with changes in light intensity and duration, cultural practices, and other environmental conditions.

I claim:

1. A new and distinct variety of floribunda rose plant named ‘KORpoplol’ characterized by the following combination of characteristics:
(a) abundantly and substantially continuously forming orange-yellow-colored striped blossoms,
(b) exhibiting a bushy and compact growth habit, and
(d) forming semi-glossy, dark green foliage;
substantially as herein shown and described.
